(19) United States
(12) Patent Application Publication
(10) Pub. No.: US 2003/0220695 A1
(43) Pub. Date: Nov. 27, 2003

(54) Inter-vertebral disc prosthesis for lumbar rachis through posterior surgery thereof

(76) Inventor: Lionel C. Sevrain, West Palm Beach, FL (US)

Correspondence Address: OGILVY RENAULT, 1981 MCGILL COLLEGE AVENUE, SUITE 1600, MONTREAL, QC, H3A2Y3, CA

(21) Appl. No.: 10/396,725

(22) Filed: Mar. 26, 2003

Related U.S. Application Data
(63) Continuation of application No. PCT/CA01/01377, filed on Sep. 26, 2001.
(60) Provisional application No. 60/235,324, filed on Sep. 26, 2000.

(51) Int. Cl. A61F 2/44

(52) U.S. Cl. 623/17.16

(57) ABSTRACT

A prosthetic inter-vertebral disc for positioning in an inter-somatic space between a pair of adjacent vertebrae (V) and within a natural annulus fibrosus (12) or a remaining portion thereof, comprises a flexible elongated outer annulus member (22) and an expandable inner nucleus member (24). The outer annulus member (22) is adapted to be introduced into the inter-somatic space through a tenotomy opening (16) and to follow an inside wall of the natural annulus fibrosus (12) such as to form a closed loop therewithin with the loop defining a chamber (20). The inner nucleus member (24) is adapted to be introduced in a dehydrated state into the inter-somatic space also through the tenotomy opening (16) and within the outer annulus member (22), and to then be hydrated so as to extend in the chamber (20) peripherally up to the outer annulus member (22). Both the outer annulus member (22) and the inner nucleus member (24) are hydrogels. The outer annulus member (22) is biased outwardly when being introduced in the inter-somatic space such that it follows the inside wall of the natural annulus fibrosus (12). A free end of the outer annulus member (22) extends outwardly of the closed loop and into the tenotomy opening for sealing the same. A method of installing the prosthetic disc is also disclosed.

### BACKGROUND OF THE INVENTION

1. Field of the Invention

The present invention relates to a synthetic inter-vertebral disc prosthesis for insertion into the rachis, for instance posteriorly into the lumbar rachis, to repair a degenerated natural disc of the spine.

2. Description of the Prior Art

U.S. Pat. No. 5,171,280 issued on Dec. 15, 1992 to Baumgartner discloses an inter-vertebral prosthesis which includes a coiler body able to rotate onto a fixed base with a flexible elastic hollow body extending from the coiler body and adapted to receive therein a filling medium through a valve. The prosthesis, once implanted and filled with an incompressible medium, is able to absorb radial forces exerted upon the periphery via the incompressible medium in the elastic hollow body. The prosthesis can be inserted in the inter-vertebral region through a small opening.

U.S. Pat. No. 3,875,595 issued on Apr. 8, 1975 to Froning discloses an inter-vertebral disc prosthesis in the form of a collapsible plastic bladder-like member which has the shape of the nucleus pulposis of a natural inter-vertebral disc. After removal of the degenerated natural nucleus pulposis, the prosthesis, in its collapsed position, is inserted through a stem and into the inter-somatic space, and a filling medium is then inserted through the stem and into the prosthesis to inflate it to a natural form. The stem is then severed just upstream of a valve thereof such that the valve remains implanted with the prosthesis.

U.S. Pat. No. 4,772,287 and No. 4,904,260 which issued respectively on Sep. 20, 1998 and Feb. 27, 1990 both in the names of Ray et al. describe the implantation of two prosthetic disc capsules side-by-side into a damaged disc of a human spine.

U.S. Pat. No. 5,192,326 and No. 5,047,055 which issued respectively on Mar. 9, 1993 and Sep. 10, 1991 both in the name of Bao et al. teach a prosthetic nucleus adapted to be implanted in the inter-somatic space of a spine and which is formed of a multiplicity of hydrogel beads which are covered by a semi-permeable membrane. This prosthetic nucleus is adapted to conform, when hydrated, to the general shape of the natural nucleus. The prosthetic nucleus is surrounded by the natural annulus fibrous. Vertebral end plates cover the superior and inferior faces of the prosthetic nucleus.

U.S. Pat. No. 4,863,477 issued on Sep. 5, 1989 to Monson discloses a synthetic inter-vertebral disc prosthesis which is made of two halves which, after having been joined together, are implanted in the inter-somatic space in place of a removed natural disc. A fluid, such as a saline solution, is then injected into the interior cavity of the prosthesis to provide the required amount of resiliency in the disc prosthesis thereby restoring proper vertebral spacing and facilitating flexibility of the spine.

U.S. Pat. No. 5,976,186 issued on Nov. 2, 1999 to Bao et al. discloses a hydrogel inter-vertebral disc nucleus adapted to be inserted in the inter-somatic space through an opening in the natural annulus for replacing the natural nucleus. The hydrogel disc is adapted to essentially fill the inter-vertebral nuclear disc cavity upon absorbing sufficient water from the body fluids.

### SUMMARY OF THE INVENTION

It is therefore an aim of the present invention to provide a novel inter-vertebral disc prosthesis.

It is also an aim of the present invention to provide an inter-vertebral disc prosthesis adapted to be installed in the inter-somatic space through posterior surgery of the rachis and, more particularly, of the lumbar rachis.

Therefore, in accordance with the present invention, there is provided a prosthetic inter-vertebral disc for positioning in an inter-somatic space between a pair of adjacent vertebrae and within a natural annulus fibrosus or a remaining portion thereof, comprising an elongated outer annulus member and an inner nucleus member, said outer annulus member being flexible and being adapted to be introduced into the inter-somatic space through a tenotomy opening and to follow an inside wall of the natural annulus fibrosus such as to form a substantially closed loop therewithin, said loop defining a chamber, said inner nucleus member being adapted to be introduced into the inter-somatic space through the tenotomy opening and within said outer annulus member and being adapted to extend in said chamber peripherally up to said outer annulus member.

Also in accordance with the present invention, there is provided a method of installing a prosthetic inter-vertebral disc in an inter-somatic space, devoid of a natural nucleus pulposus, between a pair of adjacent vertebrae and within a natural annulus fibrosus or a remaining portion thereof, comprising the steps of: (a) introducing an elongated flexible outer annulus member into the inter-somatic space through a tenotomy opening such that said outer annulus member follows an inside wall of the natural annulus fibrosus; (b) introducing an inner nucleus member in a first position thereof through the tenotomy opening and within said outer annulus member; and (c) displacing said inner nucleus member to a second position thereof such that it extends outwardly up to said outer annulus member.

Further in accordance with the present invention, there is provided a method of installing a prosthetic inter-vertebral disc in an inter-somatic space, devoid of a natural nucleus pulposus, between a pair of adjacent vertebrae and within a natural annulus fibrosus or a remaining portion thereof, comprising the steps of: (a) introducing an elongated flexible outer annulus member into the inter-somatic space through a tenotomy opening and using a mid-portion of said outer annulus member as a leading end until said outer annulus member applies against an inside wall of the natural annulus fibrosus; (b) introducing an inner nucleus member in a first position thereof through the tenotomy opening and within said outer annulus member; and (c) displacing said inner nucleus member to a second position thereof such that it extends outwardly up to said outer annulus member.

### BRIEF DESCRIPTION OF THE DRAWINGS

Having thus generally described the nature of the invention, reference will now be made to the accompanying drawings, showing by way of illustration a preferred embodiment thereof, and in which:

FIGS. 1.1 and 1.2 are top plan and lateral side views of a rachis, showing an inter-vertebral natural disc;

FIGS. 1.3 and 1.4 are top plan and lateral side views of the inter-vertebral disc of FIGS. 1.1 and 1.2 shown after removal of the natural nucleus thereof;

FIGS. 2.1 and 2.2 are top plan and lateral side views showing a prosthetic annulus of a prosthetic disc in accordance with the present invention, shown in a first position thereof;

FIGS. 3.1 and 3.2 are top plan and lateral side views similar to FIGS. 2.1 and 2.2 but showing the prosthetic annulus in a subsequent second position thereof;

FIGS. 4.1, 4.2 and 4.3 are top plan, lateral side and vertical cross-sectional views showing the prosthetic annulus as in FIGS. 3.1 and 3.2 and further showing a prosthetic nucleus of the prosthetic disc of the present invention shown in a first position thereof;

FIGS. 5.1, 5.2, 5.3 and 5.4 are top plan, lateral side and a pair of sequential cross-sectional views similar to FIGS. 4.1, 4.2 and 4.3 but showing the prosthetic nucleus in intermediate and final, i.e. hydrated, positions thereof; and

FIGS. 6.1 and 6.2 are schematic top plan and lateral side views similar to FIGS. 5.1 and 5.2 but showing, in a subsequent step, the prosthetic annulus in its final position, i.e. with the ends thereof cut.

### DESCRIPTION OF THE PREFERRED EMBODIMENTS

Generally, when an intervertebral disc must be removed from between two adjacent vertebrae, e.g. in the lumbar spine, it is less invasive to surgically proceed posteriorly from the back of the patient although the spinal cord is in the way as opposed to anteriorly which requires that surgery extends through various organs but which provides greater access to the disc. As a small incision is sufficient to proceed with the curettage of the inter-somatic space (i.e. the space between the two adjacent vertebrae) for removing the disc's nucleus, posterior surgery is preferred for this removal. On the other hand, space is limited to provide a replacement disc. Therefore, the present invention proposes a novel disc prosthesis which can be slid through the aforementioned incision and positioned in the inter-somatic space, such that the removal of the damaged disc and the installation of its prosthetic replacement are done in the same posterior operation.

The prosthesis is adapted to have a contour, once installed in the inter-somatic space, that mates with, or follows, the inner surface of the remaining part of the natural disc after the curettage thereof, e.g. in the shape of half of a cylinder with the curved section being located anteriorly and the straight section posteriorly. The straight section may be reinforced opposite the incision to prevent it from “herniating” or coming out from the inter-somatic space through the tenotomy incision once the prosthesis is under load. The anterior section may be thicker to respect the normal lordosing shape of the lumbar column, while also providing opposition to the prosthesis' tendency to move rearwardly or posteriorly. A bone substitute may be provided between the vertebrae to ensure, as time goes by, the anchoring of the prosthesis to the adjacent vertebrae.

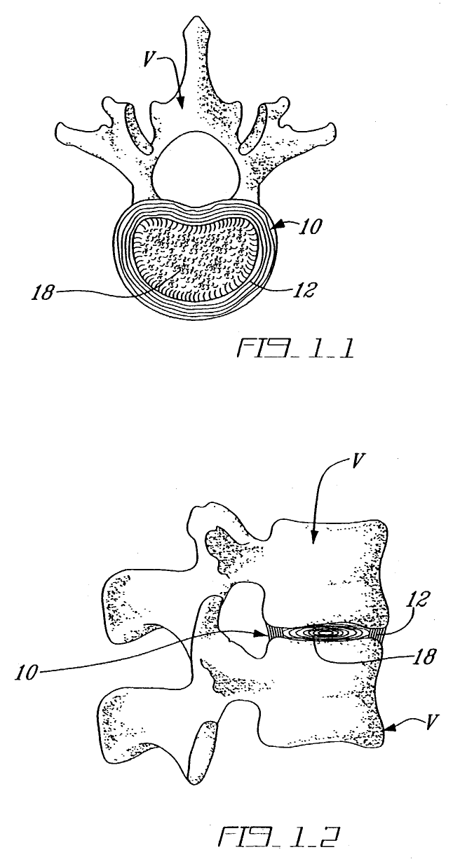

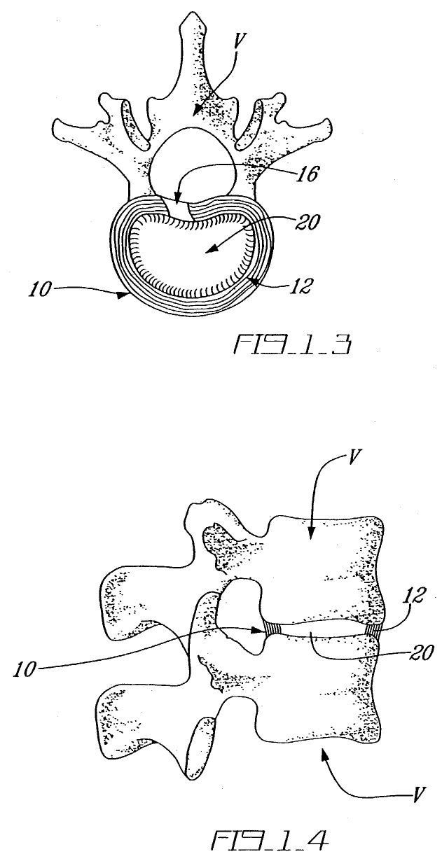

FIGS. 1.1 and 1.2 illustrate in top plan and lateral side views a disc 10 located in an inter-somatic space defined between a pair of vertebrae V. The disc 10 consists of a natural annulus fibrosus 12 and of a natural nucleus pulposus 18.

To repair a damaged disc, a tenotomy opening 16 is defined through the natural annulus 12 and the natural nucleus pulposus 18 is surgically removed thereby resulting in the disc 10 as shown in FIGS. 1.3 and 1.4. During this surgical procedure, it is possible for the natural annulus fibrosus 12 to be inwardly curetted but at least part of this annulus fibrosus 12 is retained. Therefore, as seen in FIGS. 1.3 and 1.4, a cavity 20 is defined in the inter-somatic space, inwardly of the remaining annulus fibrosus 12.

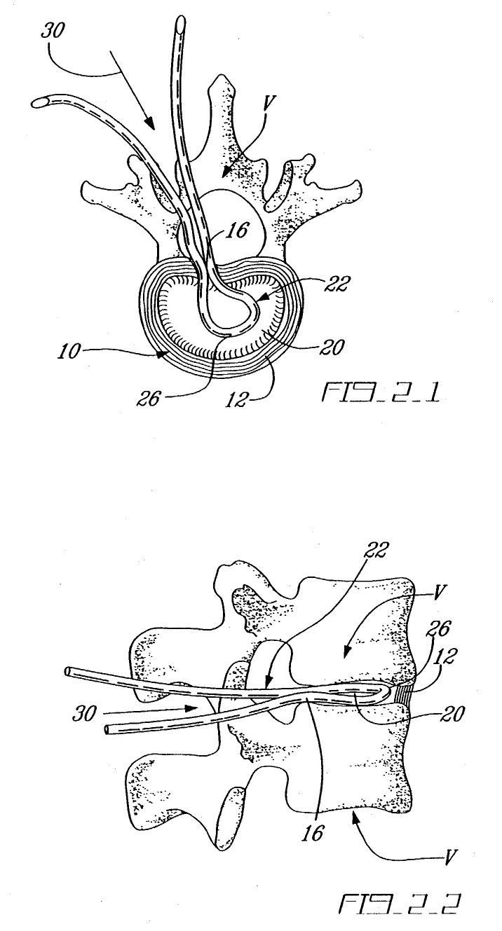

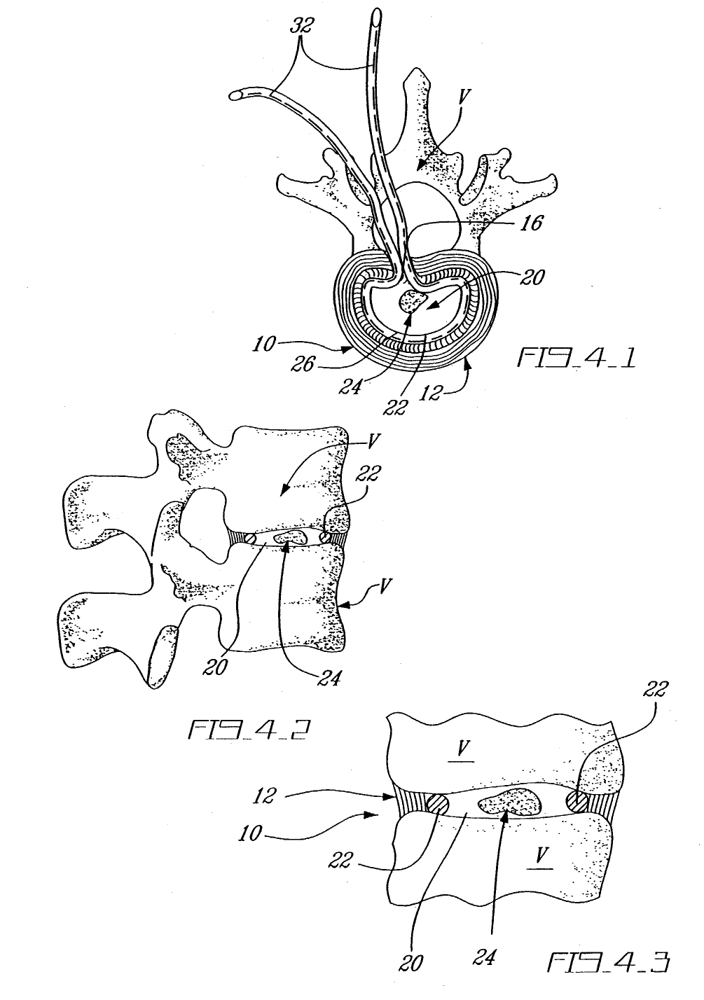

Now, in accordance with the present invention, a disc prosthesis is provided which consists of an outer annulus member and an inner nucleus member. More particularly, the annulus member takes the form of an elongated hydrogel ribbon 22 (see FIGS. 2.1 and 2.2) which may be of various shapes (e.g. cylinder, band, ribbon, prismatic, etc.), whereas the nucleus member takes the form of a hydrogel core 24 (see FIGS. 4.1 to 4.3). In FIGS. 2.1 and 2.2, the annulus ribbon 22 is inserted into the cavity 20 by way of the tenotomy opening 16. The annulus ribbon 22, which is flexible, is able to adapt such as to fit in the shape of the periphery of the inter-somatic cavity 20. For instance, it could be outwardly biased, i.e. towards an uncoiled attitude, such as to follow the inner surface of the remaining natural annulus fibrosus 12. Alternatively, the annulus ribbon 22 could be pre-formed in the shape of the periphery of the inter-somatic cavity 20.

The annulus ribbon 22 may be inserted in the intersomatic space, i.e. in the cavity 20, using various ways. The size of the annulus ribbon 22 depends on the height of the intersomatic space and on the size of the tenotomy opening 16.

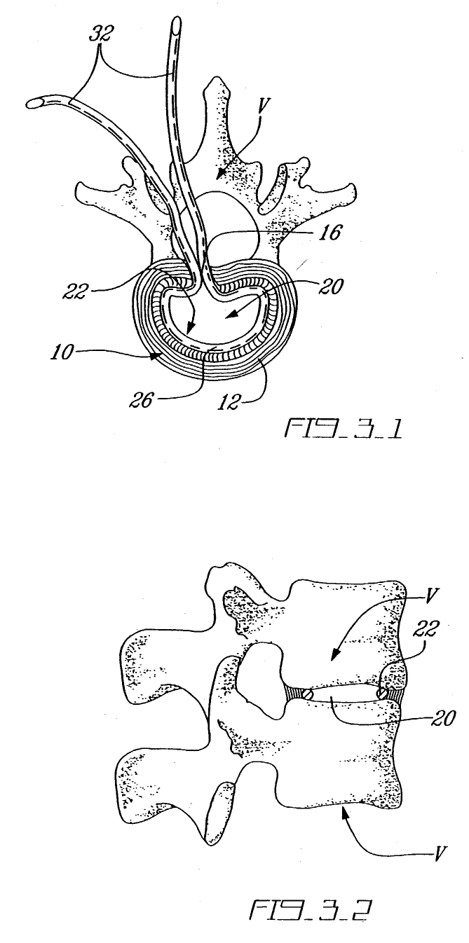

For instance, the annulus ribbon 22 may be inserted by its middle gently through the tenotomy opening 16 to allow both ends of the ribbon 22 to remain outside of the cavity 20. FIGS. 2.1 and 2.2 show the middle portion of the annulus ribbon 22 inserted in the cavity 20. A blunt spatula acting on the middle portion, i.e. the anterior curved section, of the annulus ribbon 22 is used to introduce the same along arrow 30 through the tenotomy opening 16 and into the cavity 20. By using this middle portion as a leading portion 26, both ends 32 of the annulus ribbon 22 remain outside of the intersomatic space. In FIGS. 3.1 and 3.2, the annulus ribbon 22, by its further insertion in the cavity 20, follows the complete periphery of the cavity 20. Both the ends of the annulus ribbon 22 are located side-by-side within the tenotomy opening 16 and then extend outwardly of the disc 10.

Alternatively to the method shown in FIGS. 2.1, 2.2, 3.1 and 3.2, the annulus ribbon 22 can also be inserted in the cavity 20 as follows (not shown). A leading end of the annulus ribbon 22 is inserted through the tenotomy opening 16 and into the cavity 20. Then, the annulus ribbon 22 is further gently pushed such that the leading or distal end of the annulus ribbon 22 gradually displaces along the inner surface of the annulus fibrosus 12, as the annulus ribbon 22 is slowly inserted in the disc 10 through the tenotomy opening 16.

The distal end of the annulus ribbon 22 may be larger, such as by having a bulb-like shape, to facilitate the introduction of the annulus ribbon 22 within the cavity 20 and its sliding displacement along the natural annulus fibrosus 12 by preventing the annulus ribbon 22 from being intercepted by surface irregularities that may be defined on the inner side of the natural annulus fibrosus 12.

The annulus ribbon 22, by its continuous insertion in the cavity 20, would then follow the complete periphery of the cavity 20 and would have its distal end located outwardly of the disc 10, having been passed through the tenotomy opening 16. In such a position, both the distal end and the trailing end of the annulus ribbon 22 are located side-by-side within the tenotomy opening 16 and then extend outwardly of the disc 10.

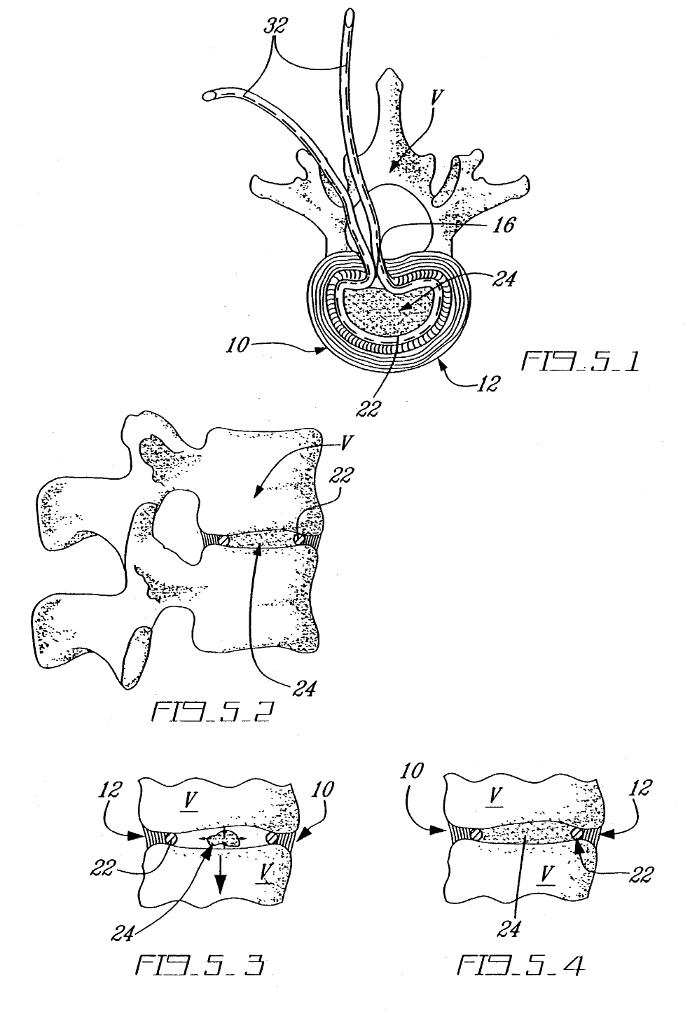

Once the annulus ribbon 22 has been properly positioned in the cavity 20 using, for instance, one of the two above methods (i.e. the first method described hereinbefore and illustrated in FIGS. 2.1, 2.2, 3.1 and 3.2, or the second, non-illustrated, method described hereinabove), the hydrogel nucleus core 24, in a dehydrated state thereof, is inserted through the tenotomy opening 16, between the two free ends of the annulus ribbon 22, and is positioned inwardly of the annulus ribbon 22 located within cavity 20, as seen in FIGS. 4.1 to 4.3. Thereafter, a fluid such as water is delivered into the cavity 20 to hydrate the nucleus core 24 which swells (see FIG. 5.3) and thus extends outwardly until it contacts the complete inner periphery of the annulus ribbon 22 located in the inter-somatic space (see FIGS. 5.1, 5.2 and 5.4).

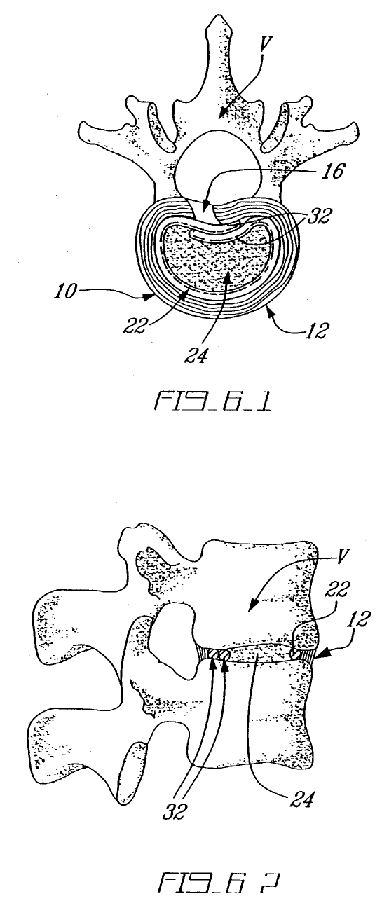

Then, the two ends 32 of the annulus ribbon 22 are cut and tucked within the cavity 20 such as to close the tenotomy opening 16 and to form a closed annulus loop in the inter-somatic space, as shown in FIGS. 6.1 and 6.2, which prevents hydrogel nucleus herniation.

The volume, and other measurements, of the cavity 20 may be evaluated prior to the installation of the present disc prosthesis such that proper annulus ribbon 22 and nucleus core 24 can be selected. The cavity volume could be, for instance, measured by introducing a fluid (e.g. water) therein, until the cavity 20 is filled therewith, and by then withdrawing the fluid from the cavity 20 by way of a syringe thereby substantially exactly measuring the cavity's volume.

The annular ribbon 22 of the present disc prosthesis is made of a first hydrogel which is non-degradable and the polymer network of which is chemically reticulated by covalent bonds. This first hydrogel is formed by free-radical solution crosslinking polymerization of at least two hydrophilic monomers from the group of dihydroxyalkyl methacrylates (when R is a methyl group of CH2=C—R) or acrylates (when R is hydrogen) such as glyceryl methacrylate (GMA) and from the group of epoxidized alkyl methacryalte or acrylates, such as glycidyl methacrylate (GdMA). GMA is used in higher proportion than GdMA, preferably within the range of from ratio of 1:0.15 and 1:0.4. The addition of a hydrophobic monomer such as a copolymerizable methacrylate or acrylate ester with an alky group preferably of 1 to 5 carbon atoms, preferably a comonomer with a ter-butyl substituent, is intended to reinforce the copolymer network to come within the scope of the present embodiment, by the introduction of hydrophobic interactions between chain segments. The hydrogel is formed in presence of a cross-linking agent which can be a glycol dimethacrylate with one ethylene group (CH₂CH₂O) or preferably polyethyelene glycol dimethacrylate with CH₂CH₂O repeat unit, or other glycol dimethacrylate monomers. The presence of multiple CH₂CH₂O groups allow optimum reactivity of unsaturated vinyl groups of monomers in presence and permit enhanced crosslinking effectiveness. To initiate the polymerization, any of the known redox initiator systems for free radical solution polymerization can be used. These include ammonium persulfate and sodium methabisulfite, or ammonium persulfate and ascorbic acid, or ammonium persulfate and sodium thiosulfate, and the like, in a proportion in amounts of 0.37 to 2% by weight. The polymerization is generally carried out at temperatures of 30° C. to about 80° C., preferably at temperatures of 40° C. to about 60° C. for 12 hours. The polymerization is conducted in a cylindrical mold with dimension suitable to replace the annulus of the disc coated with an inert material that will not react with the polymerization mixture such as polytetrafluoroethylene or polypropylene, glass, steel or aluminium. After polymerization the gel has the form of a cylinder and is washed in distilled water to attain its swelling equilibrium at 37° C. which is the physiological temperature of the body.

The hydrogels from the polymers disclosed herein are transparent and elastic and have an inflating or swelling capability in aqueous solution of 5 to 15% at equilibrium (WG). The mechanical properties of this hydrogel are of the closer of 3.54 N/mm for the horizontal tension modulus, depending on the reticulation degree of the network and/or the polymerization with an hydrophobic monomer of the group of metacrylate and acrylate ester. The hydrogel is synthetized in a mould adapted to form strips or ribbons of a height h substantially equal to a height h′ of the circumference of the inter-vertebral cavity after removal of the disc (as at FIGS. 1.3 and 1.4). The first hydrogel is sufficiently supple to be slid through the tenotomy opening 16 and will occupy a preselected volume within the inter-vertebral cavity 20.

In U.S. Pat. No. 4,056,496, there is disclosed a method to prepare hydrogels with dihydroxyalkyl acrylate and epoxidized alkyl acrylate for the formation of contact lenses. The method refers to what is called “bulk polymerization”, i.e., polymerization carried out in the absence or substantial absence of a solvent or dispersing agent, while for the purpose of the present embodiment the monomers are dispersed in a substantial amount of solvent with respect to the total weight of the monomers and free radical are formed in such a medium to initiate the polymerization reaction.

As to the nucleus core 24 of the present disc prosthesis, it is made of a second hydrogel, which is non-biodegradable and the polymer network of which is chemically reticulated by covalent bonds, and which has visco-elastic properties that are similar to those of the natural nucleus pulposus 18 such as to counterbalance or offset the external hydrostatic pressure which is exerted thereon. This second hydrogel has a swelling or inflating capability in an aqueous solution of about 60 to 85%, at equilibrium (WG). The hydrogel is made of a co-polymer of glyceryl methacrylate and glycidyl methacrylate, crosslinked with a glycol dimethacrylate with one ethylene group (CH₂CH₂O) or preferably polyethyelene glycol dimethacrylate with CH₂CH₂O repeat unit, or other glycol dimethacrylate monomers. By varying the proportion of the glyceryl methacrylate and the glycidyl methacrylate, and in the presence of a crosslinking agent, the inflating capability increases to resemble that of the nucleus. In addition, the expansion factor, as measured by the Swelling Ratio [SR=(d₁−d₀)/ d₀, where d₀ is the diameter of the dried gel and d₁ the diameter of the swollen gel, as prepared to form a disc] upon swelling equilibrium has to be taken into account in the final size of the gel so that to fill the inter-vertebral space containing the first hydrogel annulus and to contact by adhesive forces the inner periphery of the annulus ribbon 22 (FIGS. 5.1, 5.2 and 5.4).

The ratio of GMA to GdMA varies within the range of from 1:0.06 to 1:0.2.

Both gels are sterilized by autoclaving prior use at 121° C. for 30 minutes.

### EXAMPLE 1

First hydrogel of the disc prosthesis

The mixture of 88.77% by weight of GMA, 8% by weight of GdMA, 2% by weight of ter-butyl methacrylate, 1.23% by weight of ethylene glycol dimethacrylate is dissolved in distilled water to produce a solution containing 40% by weight of solute. Ammonium persulfate (6% v/v) and sodium metabisulfite (12% v/v) were added in a 0.37% ratio to GMA. The mixture was transferred in a glassware put in low temperature bath and the solution was bubbled 15 minutes through with pure nitrogen. The glassware was sealed and placed in a constant temperature bath at between 55° C. and 60° C. Polymerization was allowed to proceed for 12 hours. After the polymerization is completed, a clear and rigid gel is obtained and is soaked in distilled water with frequently for extensive washing to remove unreacted products.

### EXAMPLE 2

The procedure of Example 1 is repeated except that the amount of GMA is 90.77% by weight and the amount of GdMA is 6% by weight.

### EXAMPLE 3

Second Hydrogel

The mixture of GMA and GdMA in proportion of 2 % by weight of GdMA with respect to the GMA with 1.23% by weight of ethylene glycol dimethacrylate is dissolved in distilled water to produce a solution containing 30% by weight of solute. Polymerization is initiated as in Example 1.

### EXAMPLE 4

Example 1 is repeated except that the crosslinking agent is tetraethylene glycol dimethacrylate.

The second hydrogel is dehydrated and is manually introduced under visual control in the inter-vertebral cavity 20, i.e. at the location of the former natural nucleus pulposus 18 (see FIGS. 4.1 to 4.3), and is then re-hydrated in an aqueous solution until its maximal swelling capability (W_{G}), as in FIGS. 5.1, 5.2 and 5.4. The second hydrogel is prepared in such a way that WG corresponds to a pre-selected specific volume of the inter-vertebral cavity 20 after removal of the natural disc. The adhesive properties of the second hydrogel allows it to adhere to the facing vertebral surfaces of the upper and lower vertebrae V between which it is located in the inter-somatic space, and also to adhere to the first hydrogel constituting the annulus ribbon 22.

The first hydrogel that forms the annular ribbon 22 should substantially reproduce as close as possible the rigidity characteristics of the natural annulus fibrosus in order to reinforce the discal annular belt, while being able to efficiently seal the tenotomy opening 16 to prevent the annular ribbon 22 from herniating. As to the second hydrogel forming the nucleus core 24, should as much as possible have the deformation properties and the coherence characteristics of the natural nucleus pulposus in order to respectively have dampening curves compatible with the typical levels of mechanical loads of natural lumbar discs and have resistance to fracturing under applied pressures.

The hydrogel annulus ribbon 22 may, as mentioned hereinbefore, have an intrinsic resiliency, or memory, that gives it a tendency to straighten out such that it is biased outwardly during its displacement in the cavity 20 and so maintains contact with the natural annulus fibrosus 12.

Alternatively, the nucleus hydrogel might be shaped in a series of independent flexible micro-beads (e.g. spheres containing appropriate fluid for damping effect) which would be easily insertable through the tenotomy incision and into the inter-somatic space.

1. A prosthetic inter-vertebral disc for positioning in an inter-somatic space between a pair of adjacent vertebrae and within a natural annulus fibrosus or a remaining portion thereof, comprising an elongated outer annulus member and an inner nucleus member, said outer annulus member being flexible and being adapted to be introduced into the inter-somatic space through a tenotomy opening and to follow an inside wall of the natural annulus fibrosus such as to form a substantially closed loop therewithin, said loop defining a chamber, said inner nucleus member being adapted to be introduced into the inter-somatic space through the tenotomy opening and within said outer annulus member and being adapted to extend in said chamber peripherally up to said outer annulus member.

2. A prosthetic inter-vertebral disc as defined in claim 1, wherein said outer annulus member and said inner nucleus member are respectively made of first and second hydrogels.

3. A prosthetic inter-vertebral disc as defined in claim 1, wherein when said prosthetic disc has been installed, said closed loop comprises a pair of ends of said outer annulus member that overlap in the inter-somatic space and block the tenotomy opening for preventing hydrogel nucleus herniation.

4. A prosthetic inter-vertebral disc as defined in claim 1, wherein said outer annulus member is biased outwardly when being introduced in the inter-somatic space such that it applies on the inside wall of the natural annulus fibrosus.

5. A prosthetic inter-vertebral disc as defined in claim 1, wherein said inner nucleus member is adapted to be introduced through the tenotomy opening into the chamber in a dehydrated position thereof, and to then be hydrated such as to expand up to an inside surface of said outer annulus member.

6. A prosthetic inter-vertebral disc as defined in claim 2, wherein said outer annulus member is made of a first hydrogel which is non-degradable and the polymer network of which is chemically reticulated by covalent bonds.

7. A prosthetic inter-vertebral disc as defined in claim 2, wherein said inner nucleus member is made of a second hydrogel, which is non-biodegradable and the polymer network of which is chemically reticulated by covalent bonds, and which has visco-elastic properties that are similar to those of the natural nucleus pulposus such as to counterbalance or offset the external hydrostatic pressure which is exerted thereon.

8. A method of installing a prosthetic inter-vertebral disc in an inter-somatic space, devoid of a natural nucleus pulposus, between a pair of adjacent vertebrae and within a natural annulus fibrosus or a remaining portion thereof, comprising the steps of: (a) introducing an elongated flexible outer annulus member into the inter-somatic space through a tenotomy opening such that said outer annulus member follows an inside wall of the natural annulus fibrosus; (b) introducing an inner nucleus member in a first position thereof through the tenotomy opening and within said outer annulus member; and (c) displacing said inner nucleus member to a second position thereof such that it extends outwardly up to said outer annulus member.

9. A method as defined in claim 8, wherein said outer annulus member is adapted in step (a) to form a substantially closed loop within the natural annulus fibrosus, said loop defining a chamber, said inner nucleus member being adapted to be positioned in said chamber.

10. A method as defined in claim 8, wherein said outer annulus member and said inner nucleus member are respectively made of first and second hydrogels.

11. A method as defined in claim 9, wherein a free end of said outer annulus member is positioned such as to extend outwardly of said closed loop and into the tenotomy opening for substantially sealing the same.

12. A method as defined in claim 8, wherein said outer annulus member is biased outwardly when being introduced in step (a) in the inter-somatic space such that it follows the inside wall of the natural annulus fibrosus.

13. A method as defined in claim 8, wherein said inner nucleus member, in step (b) , is introduced through the tenotomy opening in a dehydrated position thereof; and to then be, in step (c), hydrated such as to expand up to an inside surface of said outer annulus member.

14. A method of installing a prosthetic inter-vertebral disc in an inter-somatic space, devoid of a natural nucleus pulposus, between a pair of adjacent vertebrae and within a natural annulus fibrosus or a remaining portion thereof, comprising the steps of: (a) introducing an elongated flexible outer annulus member into the inter-somatic space through a tenotomy opening and using a mid-portion of said outer annulus member as a leading end until said outer annulus member applies against an inside wall of the natural annulus fibrosus; (b) introducing an inner nucleus member in a first position thereof through the tenotomy opening and within said outer annulus member; and (c) displacing said inner nucleus member to a second position thereof such that it extends outwardly up to said outer annulus member.

15. A method as defined in claim 14, wherein said outer annulus member is adapted in step (a) to form a substantially closed loop within the natural annulus fibrosus, said loop defining a chamber, said inner nucleus member being adapted to be positioned in said chamber.

16. A method as defined in claim 14, wherein said outer annulus member and said inner nucleus member are respectively made of first and second hydrogels.

17. A method as defined in claim 14, wherein a pair of ends of said outer annulus member are positioned in an overlapping way in the inter-somatic space such that said closed loop closes off the tenotomy opening for substantially sealing the same.

18. A method as defined in claim 14, wherein said outer annulus member is biased outwardly when being introduced in step (a) in the inter-somatic space such that it applies against the inside wall of the natural annulus fibrosus.

19. A method as defined in claim 14, wherein said inner nucleus member, in step (b), is introduced through the tenotomy opening in a dehydrated position thereof; and to then be, in step (c), hydrated such as to expand up to an inside surface of said outer annulus member.
